(19) United States
(12) Patent Application Publication
(10) Pub. No.: US 2006/0229540 A1
(43) Pub. Date: Oct. 12, 2006

(54) Finger splint & bandage cover

(76) Inventor: Rosie Sharon Atwood, Salt Lake City, UT (US)

Correspondence Address: ROSIE YAMAGUCHI, 1777 SOUTH 300 EAST, SALT LAKE CITY, UT, 84115, US

(21) Appl. No.: 11/025,207

(22) Filed: Apr. 7, 2005

(51) Int. Cl. A61F 5/00

(52) U.S. Cl. 602/22

(57) ABSTRACT

My invention, the Finger Splint and Bandage Cover is devised to protect and secure the injured finger/thumb and keep the splint and bandage in place. It is designed to protect the finger/thumb from exposure such as dirt, grease, oil, etc. Is comprised of nylon/cotton blend material. Designed to fit over the splint/bandage using a continuous piece of strip from the finger/thumb to the wrist. When attached and wrapped around the wrist the covering would be secured to the hand with “VELCRO”, i.e., hoops and loops. Cover can come in various sizes and colors.

### CROSS-REFERENCE TO RELATED APPLICATIONS

Not Applicable

### STATEMENT REGARDING FEDERALLY SPONSORED REARCH OR DEVELOPMENT

Not Applicable

### THE NAMES OF THE PARTIES TO A JOINT RESEARCH JOINT AGREEMENT

Not Applicable

### INCORPORATION-BY-REFERENCE OF MATERIAL SUBMITTED ON A COMPACT DISC

Not Applicable

### BACKGROUND OF THE INVENTION

1. Field of the Invention

My sister had sprung her finger while at work. She had on this ugly splint which covered her injury and her splint was not secure enough. I thought I would make a covering that could fit over her splint to secure it better and by covering the splint would make her injured finger more attractive to look at. Also, my son who was a mechanic injured his finger and the bandage he wore kept slipping off while he worked. I made him a cover to fit over his bandaged finger.

The cover aided tremendously by keeping the bandage in place and kept oil, grease and dirt from getting underneath his bandage thus keeping the injury more sterile.

2. Description of Related Art

As depicted in the art art drawing, the cover is devised as a partial glove illustrating the covering of the injured finger. Depending on which finger is injured it would cover the splint/bandage of the injured finger leaving all other fingers and thumb exposed. A strap attached with “VELCRO”, i.e. hoops and loops, would wrap around the wrist forming a wristband to secure the glove-like covering keeping the splint/bandage in place.

### BRIEF SUMMARY OF THE INVENTION

The Finger Splint and Bandage Cover would greatly benfit the wearer. Comprised of a nylon/cotton blend material would easily slip over a splint or injured bandage finger/thumb. This cover would provide more protection to the injury and help prevent the splint/bandage from slipping off. The use of this cover would conceal the splint/bandage from being an eyesore and the hand would present a more attractive appearance.

### BRIEF DESCRIPTION OF THE SEVERAL VIEWS OF THE DRAWING

Palm-side up for the Finger Splint and Bandage Cover.

FIG. 1A illustrates a slot which will cover the full length of the splint/bandage on the injured finger. which connects to the palm-side down on drawing #2. FIGS. 2A and 3A is part of palm-side down.

FIG. 2A illustrates the base of the finger to the top of the wrist a continuous single-layer strip, the width of the finger/thumb.

FIG. 3A illustrates a single-band strip, forming a wristband, which wraps around the wrist secured with “VELCRO”, i.e., hoops and loops.

FIG. 4A illustrates the placement of the “VELCRO”, i.e., hoops and loops on the wristband which is located on palm-side up.

Drawing #2 palm-side down mirrors palm-side up. Below the continuous strip from the bottom of the finger to the wristband “VELCRO”, i.e., hoops and loops will connect to palm-side up on the single-strip wristband.

### DETAILED DESCRIPTION OF THE INVENTION

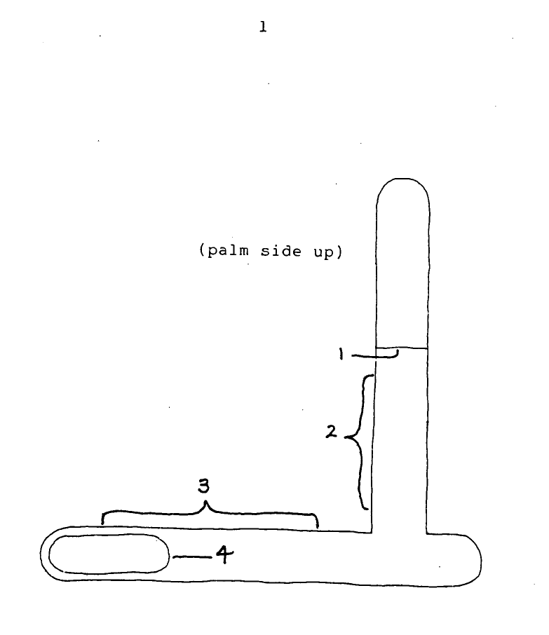

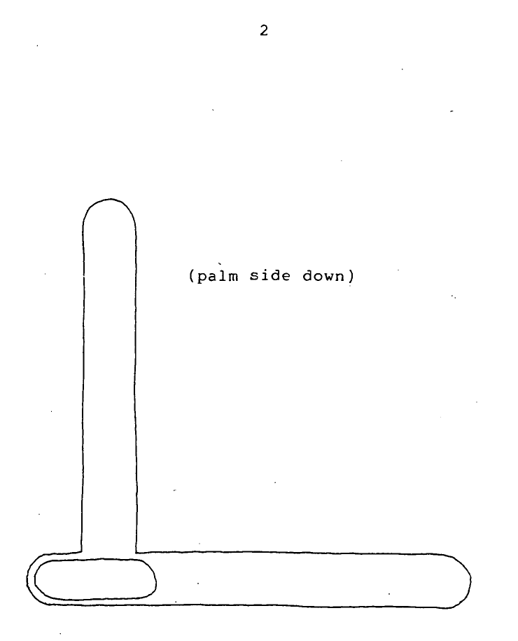

The Finger Splint and Bandage Cover, made out of nylon/cotton blend material would be stretchable and breathable. The injured finger/thumb with splint/bandage on would slip into the open slot as described in FIG. 1A, palm-side up and connected to palm-side down. As noted in drawing #2 is a continuous single-layer strip, palm-side down (backside of hand) which starts from the base of the finger/thumb and is measured for size using the width of the finger or thumb. The continuous single-layer strip is then connected to the wristband securing the splint/bandage in place. FIG. 3A depicts a single strip, continuing on palm-side down and is connected to a single strip forming a wristband. “VELCRO”, i.e., hoops and loops are located at the end of the strip as noted in FIG. 4A, palm-side up. Drawing #2, Palm-side Down. Just below the continuing strip, from the base of the finger to the top of the wrist on the single-layer strip which forms a wristband is “VELCRO”, i.e., hoops and loops which is located on the end of the strip. When the continuous strip and wristband are connected together the finished product would have a glove-like appearance but would not cover the whole hand. Only the splint/bandage would be covered.

What I claim as my invention is: The Finger Splint and Bandage Cover.

1. I claim that the Finger Splint and Bandage Cover can come in various sizes and colors.

2. I claim that wristband securing the Finger Splint and Bandage Cover can be secured with “VELCRO”, i.e. hoops and loops.

3. I claim that by wrapping the Finger Splint and Bandage Cover wristband around the wrist would secure the existing splint and or bandage from slipping off.

4. I claim that the Finger Splint and Bandage Cover would be a one continuous piece that would have a glove-like appearance covering the already existing splint and or bandaged injury.

5. I claim that the cover will fit over the already existing splint or bandaged injured finger or thumb and can come in various sizes as stated in claim #1.

6. I claim that material used be made of stretchable and breathable material, i.e., nylon/cotton blend to accommodate the Finger Splint and Bandage Cover.

7. I claim that the use of nylon/cotton blend material would stretch over the already existing splint or bandaged finger or thumb.

8. I claim that wearing the Finger Splint and Bandage Cover will help aid in protecting the already existing splint or bandaged finger or thumb from, i.e., dirt, grease, oil and the wear of this cover would present a more attractive appearance when placed over the splint or bandage.
